(19) United States
(12) Patent Application Publication
(10) Pub. No.: US 2015/0328166 A1
(43) Pub. Date: Nov. 19, 2015

(54) Device for the Treatment of Bone Conditions

(71) Applicant: ActiveSignal Holding Limited, London (GB)

(72) Inventor: Ward Warren, Auckland (GB)

(21) Appl. No.: 14/812,295

(22) Filed: Jul. 29, 2015

Related U.S. Application Data
(63) Continuation of application No. 14/274,182, filed on May 9, 2014.

(30) Foreign Application Priority Data
May 14, 2013 (NZ) NZ610572
Jul. 26, 2013 (AU) 2013209343

(51) Int. Cl. A61K 9/48; A61K 9/28; A61K 33/10; A61K 33/00; A61K 33/42; A61K 33/14

(52) U.S. Cl. CPC A61K 9/4891; A61K 33/42; A61K 33/14; A61K 33/10; A61K 33/00; A61K 9/28

(57) ABSTRACT

A composition comprising at least one substance coated with an agent wherein the agent forms a liquid impermeable but gas permeable layer surrounding the said substance and preventing the passage of the said substance through said coating, wherein in-use the said substance is not released into solution in the body, and wherein said substance is selected from one or more of the group consisting of calcium phosphate, sodium chloride, potassium chloride, magnesium chloride, calcium carbonate, and sodium bicarbonate for use in the treatment of osteoporosis and other bone conditions.

### CROSS REFERENCE TO RELATED APPLICATIONS

This application claims priority to the following patent applications: (1) U.S. patent application Ser. No. 14/274,182 filed May 9, 2014; (2) New Zealand Patent Application NZ 610572, filed May 14, 2013; and (3) Australian Patent Application 2013209343, filed Jul. 26, 2013; each of the above cited applications is hereby incorporated by reference herein as if fully set forth in its entirety.

The present invention relates to the field of prevention and treatment of the debilitating condition of bones known as osteoporosis and other bone conditions. In particular the invention relates to a device including a salt selected from sodium chloride, potassium chloride, magnesium chloride, sodium bicarbonate, calcium carbonate, calcium phosphate and combinations thereof, for the treatment of osteoporosis.

### DESCRIPTION

Osteoporosis is a chronic progressive condition and the most common human metabolic bone condition prevalent in the United Kingdom and the United States. It has been most frequently recognised in older white women, but also occurs in both genders, all races and all age groups.

Osteoporosis is a systemic chronic skeletal condition characterised by loss of bone mass and micro-architectural deterioration of bone tissue, that leads to enhanced bone fragility and a consequent increase in the risk of fractures. Osteoporosis represents an increasingly serious health and economic problem in the United Kingdom, the United States, and around the world.

The cost to the UK National Health Service of osteoporotic hip fractures alone is over two billion pounds per year [Protecting fragile bones. National Osteoporosis Society] The equivalent amount for the United States is about 19 billion dollars per year Osteoporosis often does not become clinically apparent until a bone fracture occurs. Many individuals, male and female, experience pain, disability, and continuing diminished quality of life as a result of having osteoporosis. It is estimated that over one third of post-menopausal women are at risk of osteoporosis.

It is known that post-menopausal osteoporosis in women arises from changes in hormone levels, principally the hormones oestrogen and progesterone. Oestrogen therapy is known to improve outcome in post-menopausal women with osteoporosis. However oestrogen therapy with or without progesterone has been found to increase the risk of pulmonary embolism, vein thrombosis (DVT), stroke, invasive breast cancer, myocardial infarction and dementia. Because of these risks most women refuse hormone therapy.

Current treatment includes calcium and vitamin D supplementation, and advice on exercise and diet. Pharmaceutical treatments include anti-resorptive agents such as the bisphosphonates, alendronate, etidronate and risedronate, the selective oestrogen receptor modulator (SERM) raloxifene, strontium ranelate, and denosumab. One anabolic agent, teriparatide, is also available. Biphosphonates have relatively complex instructions for oral administration.

All of the pharmaceutical treatments have unpleasant side effects in most patients, making many physicians reluctant to prescribe and many patients refusing to take the treatments. The respected National Institute for Health and Clinical Excellence in the UK recommends against pharmaceutical treatment of post-menopausal women and others with osteoporosis under 65 years of age unless there is an assessed independent risk of osteoporotic fracture. (ISBN 1-84629-836-9).

Bone is living, growing tissue that constantly forms new bone while replacing older bone. Bone continuously renews and changes through a process known as remodeling. The bone remodeling cycle consists of two distinct stages: (1) bone resorption (breakdown or removal) and (2) bone formation. During resorption, osteoclast cells on the bone's surface dissolve bone tissue and create small cavities. During formation, osteoblast cells fill the cavities with new bone tissue.

Throughout life, bone resorption and bone formation normally takes place in close sequence and remains balanced. An imbalance in the bone remodeling cycle occurs with menopause in women and with ageing in both genders, and it can occur earlier in life with other conditions.

This imbalance can result in gradual bone loss that eventually leads to osteoporosis and bone fractures.

The anti-resorptive pharmaceutical treatments generally slow the bone loss that occurs in the breakdown part of the remodeling cycle, but new bone may still be made at a lower than normal rate. As a result there may only be a small increase in bone density. Teripatide, a form of parathyroid hormone, increases the rate of bone formation and is an anabolic drug, a distinct category of osteoporosis pharmaceutical.

None of the pharmaceutical treatments can be said to be a cure for osteoporosis and all have significant side effects including bone, joint and muscle pain. Side effects of oral tablets include nausea, heartburn, irritation of the oesophagus and gastric ulcer. The side effects of teripatide (given by injection) include leg cramps and dizziness.

Osteopenia is a term applied to subjects whose bone mineral density is slightly below the average range, as measured on a bone density scan.

Osteopenia is not normally treated, on the basis of a scan reading, unless there are other factors indicating treatment such as a previous fracture.

Loss of normal bone mineral density may be measured by dual energy X-ray absorptiometry, known as a DEXA scan. A DEXA scan compares the subject's reading with a young healthy adult. The difference is then calculated as a T-score standard deviation (SD). The World Health Organization classifies a T-score of above −1 SD as normal, between −1 and −2.5 SD as osteopenia, and below −2.5 SD as osteoporosis.

For the purposes of this invention the word osteoporosis is defined to include osteopenia.

Reference herein to medically efficacious substances is to substances which can be used to effect a treatment. Therefore a medically efficacious substance includes any substances which can be used to effect the therapy or prophylaxis of a condition.

Reference to treatment provided by the instant invention includes therapy, prophylaxis and maintenance of optimum bone health.

The inventor does not wish to be bound by the hypothesis, but he believes it is likely that osteoporosis commences with life events which upset the total mineral electrolyte balance throughout the body. One such event is the menopause in women where levels of progesterone are lowered. Progesterone normally helps to maintain the levels of aldosterone, the principal mineralocorticoid which regulates levels of sodium and potassium, the distribution of liquids, and the balance of electrolytes in the body. When aldosterone is lowered, reducing available sodium and potassium, the inventor believes that the body may react by inappropriate conservation or locking up of many essential minerals such as calcium, making them unavailable for the normal balance of bone remodeling.

The invention, by providing an apparent surplus of appropriate ions within the body environment in proximity to epithelial cells, prompts a body reaction releasing more than sufficient ions to reinstate effective normal ion electrical activity and signalling.

It follows that a method of creating an apparent surplus of the requisite electrolyte minerals in the body would have the effect of ensuring the full availability of the minerals needed for normal bone remodeling.

It has previously been discovered that, for example, sodium chloride coated with a gas permeable but liquid impermeable layer, is an effective treatment for exocrine gland diseases, as detailed in NZ 545365 herein incorporated by reference. This type of composition is distinctly different from pharmaceutical products since no medically efficacious substance is taken into solution in the body. This type of sealed composition is classified as a medical device.

The therapy for osteoporosis and related bone disorders provided by this invention makes a significant new contribution to the art.

Current treatments, (other than hormone treatments which have many disadvantages), seek to intervene in the process of either bone formation or bone resorption. Such selective approaches require considerable skill in finding the appropriate treatment for the patient, followed by close monitoring to judge the effect of administration of the treatment. Further, the administration of pharmaceutical products designed to change the balance of natural regulation of body systems leads to additional risks and side effects.

The instant invention employs the novel approach to the treatment of osteoporosis and related bone disorders by prompting the body system to make a small adjustment to restore the natural balance of available electrolyte ions. The result of this intervention is to restore the proper balance of total bone turnover, formation and resorption simultaneously, as exemplified by the examples provided, and with no danger of additional risks or side effects.

The invention relates to the use of sodium chloride, potassium chloride, magnesium chloride, sodium bicarbonate and calcium phosphate enclosed within a coating which is impermeable to liquid but permeable to gas, in the treatment of osteoporosis and other bone conditions in humans.

The coating may comprise enclosure within a ceramic, or an enclosing layer may be made using polymers available in the art, or a natural wax, or beeswax hardened with cornstarch and talc.

It will be appreciated that the invention (in all its aspects) is particularly useful for treating human subjects. However the subject may be any other mammal of veterinary interest.

It will be appreciated that the invention may provide useful treatment for other bone conditions in addition to osteoporosis including osteopenia, Paget's disease of bone, osteoporosis caused by medications including glucocortiocoids and occurring in connection with certain diseases such as rheumatoid arthritis and multiple sclerosis and for osteogenesis imperfecta.

It will be appreciated that the invention may provide useful treatment in any condition requiring bone growth including the repair of bone fracture and periodontal repair and regrowth.

In a first aspect, the invention provides a medically efficacious composition formulated as a medical device comprising at least calcium phosphate, preferably di-calcium phosphate, coated with a liquid impermeable but gas permeable layer.

According to a second aspect, the invention provides a medically efficacious composition formulated as a medical device comprising at least calcium phosphate, preferably di-calcium phosphate, and sodium chloride, coated with a liquid impermeable but gas permeable layer.

According to a third aspect, the invention provides a medically efficacious composition formulated as a medical device comprising at least calcium phosphate, preferably di-calcium phosphate, sodium chloride and potassium chloride, coated with a liquid impermeable but gas permeable layer.

According to a fourth aspect, the invention provides a medically efficacious composition formulated as a medical device comprising at least calcium phosphate, preferably di-calcium phosphate, sodium chloride, potassium chloride and magnesium chloride, coated with a liquid impermeable but gas permeable layer.

According to a fifth aspect, the invention provides a medically efficacious composition formulated as a medical device comprising at least calcium phosphate, preferably di-calcium phosphate, sodium chloride, potassium chloride, magnesium chloride and sodium bicarbonate, coated with a liquid impermeable but gas permeable layer.

According to a sixth aspect, the invention provides a medically efficacious composition formulated as a medical device comprising at least calcium phosphate, preferably di-calcium phosphate, sodium chloride, potassium chloride, magnesium chloride, sodium bicarbonate, and calcium carbonate coated with a liquid impermeable but gas permeable layer.

According to a seventh aspect, the invention provides a medically efficacious composition formulated as a medical device comprising at least one or more of calcium phosphate, preferably di-calcium phosphate, sodium chloride, potassium chloride, magnesium chloride, sodium bicarbonate, and calcium carbonate, coated with a liquid impermeable but gas permeable layer.

According to the eighth aspect, the invention comprises a composition formulated as a medical device comprising any of the above described medically efficacious compositions in combination with effective treatments known in the art.

According to the ninth aspect, the invention provides a composition formulated as a medical device comprising any of the above described medically efficacious compositions used alone or in combination with effective treatments known in the art for the treatment of bone conditions.

According to the tenth aspect, the invention provides a composition formulated as a medical device comprising at least one or more of calcium phosphate, preferably di-calcium phosphate, sodium chloride, potassium chloride, magnesium chloride, sodium bicarbonate, and calcium carbonate, or an acceptable salt thereof or another acceptable salt, coated with a liquid impermeable but gas permeable layer.

It should be noted that where compounds such as sodium chloride and di-calcium phosphate and others are not normally considered medically efficacious, in the context of this invention they are medically efficacious.

The composition formulated as a medical device may be combined with a preparation of at least one ingredient designed for delivery into solution, such as colouring to improve appearance, or at least one ingredient designed for delivery into solution for a therapeutic purpose.

It is an object of this invention to provide a composition in the form of a medical device for the prevention and treatment of osteoporosis and other bone conditions, which has advantages over any active substances known from prior art.

This object is achieved by the subject matter of the claims.

It is known that dietary and other mineral supplementation is recommended for persons with osteoporosis, for example calcium phosphate and calcium carbonate, combined with or without pharmaceutical treatment.

However the instant invention is distinctly different from such supplementation, and from pharmaceutical treatments, since the substances used in the invention do not enter into body circulation but are evacuated from the body intact with nothing entering circulation. The invention has the advantages of avoiding accumulation and avoiding side effects. There is nothing in the prior art which would suggest that substances which are not dissolved in the body in-use could be useful in the treatment of bone conditions.

According to an embodiment of the present invention a skin patch may be used to effect treatment of osteoporosis and other bone conditions, the patch being provided with two granules of the coated composition as described herein. The two granules may be of 2 mm to 10 mm diameter and fixed to the patch at a distance of 1 to 100 mm apart, but preferably 30 mm apart, and the patch or other mounting device, held against or near to the skin. The patch may be an adhesive patch with the granules mounted on the adhesive side.

Forms of the invention which may readily be made and used include a pill, a tablet, a lozenge, a bolus, a capsule, a caplet, a granule, and a microparticle. Granules may be used as sprinkles, and granules and microparticles may be used in a suspension, cream, gel or paste. The invention may be provided as an implant.

The use of the invention is illustrated by the following non-limiting examples.

### EXAMPLE 1

A capsule formulation of the invention as detailed above was made containing, by proportion of weight, sodium chloride 35, potassium chloride 4, magnesium chloride 2.5, calcium carbonate 2, sodium bicarbonate 5, di-calcium phosphate 15, and the excipients to harden the wax gas permeable but water impermeable layer were, cornstarch 15, magnesium silicate 7.5. All the ingredients were dried and ground to a fine powder which was then well mixed in white wax USP, by proportion of weight 250 and melted at 85 deg C. The molten mixture was then used to fill vegetable capsules size “0”.

The contents of the capsules were then tested by stirring in 0.1M hydrogen chloride water solution for 3 hours followed by stirring in phosphate buffer pH 7 water solution for 24 hours. This non-dissolution test replicating passage through the GI tract showed that the test samples were unchanged, by weighing and by visual inspection, by the test.

A further test of the contents of the capsule was made, stirring in deionised water in a closed container for 27 hours. The water was then tested for sodium and none was found indicating that no leakage from the capsule contents was occurring.

Five post menopausal women aged between 58 and 67 years were selected from subjects giving informed consent.

Each woman took one capsule per day for 30 days and no other medication. Blood samples were taken from each woman on day 0 and day 30.

The blood samples taken were analysed for the well-known and accepted bone resorption marker assay based on specific antibodies raised against isolated collagen peptides containing cross-links, in this case the beta-isomer of C-telopeptide of type 1 collagen known as CTX Crosslaps.

The lab results for each numbered subject were as follows:
1. Day0 0.410 Day30 0.283 reduction −32%
2. Day0 0.333 Day30 0.216 reduction −35%
3. Day0 0.261 Day30 0.188 reduction −28%
4. Day0 0.199 Day30 0.158 reduction −20%
5. Day0 0.115 Day30 0.050 reduction −56%

The blood samples were also analysed for the bone formation rate marker, osteocalcin, a small protein of 49 amino acids.
1. Day0 27.2 Day30 26.4 reduction −3%
2. Day0 14.6 Day30 14.1 reduction −3%
3. Day0 16.6 Day30 13.6 reduction −18%
4. Day0 12.6 Day30 09.9 reduction −21%
5. Day0 09.1 Day30 07.5 reduction −17%

It is well known that moderate alcohol consumption provides some protection against osteoporosis in post-menopausal women, lowering the bone resorption marker CTX and also lowering the bone formation marker osteocalcin. [Moderate alcohol intake lowers biochemical markers of bone turnover in postmenopausal women. 2012 DOI:10.1097/gme.0b013e31824ac071]

The reduction in bone resorption marker and in bone formation marker provided by the present invention shows a far greater improvement in bone health as indicated by the two markers.

None of the five participants reported any side effects as a result of taking the capsules as described. Side effects are most unlikely since the contents of the capsules are unchanged by being used and no substance escapes into the body.

### EXAMPLE 2

A tablet formulation of the invention as detailed above was made containing, by proportion of weight, sodium chloride 35, potassium chloride 4, magnesium chloride 2.5, calcium carbonate 2, sodium bicarbonate 5, di-calcium phosphate 15, and the excipients microcrystalline cellulose 40, and lactose EP 13. These ingredients were finely ground and thoroughly mixed and compressed to form 12 mm diameter tablets.

The tablets were then thickly spray coated with the ethyl acrylate, methyl methacrylate copolymer EUDRAGIT RL30D (Evonik Industries AG, Rellinghauser Strasse 1-11, 45128 Essen, Germany), in accordance with the manufacturer's instructions, to form a polymer matrix coating with a weight of about 12 mg per tablet.

The tablets were then tested by stirring in 0.1M hydrogen chloride water solution for 3 hours followed by stirring in phosphate buffer pH 7 water solution for 24 hours. This non-dissolution test replicating passage through the GI tract showed that the test samples were unchanged, by weighing and by visual inspection, by the test.

A further test of the tablets was made, by agitating in deionised water in a closed container for 27 hours. The water was then tested for sodium and none was found, indicating that no leakage from the capsule contents was occurring.

Ten volunteer post menopausal women aged between 58 and 65, having given informed consent, and with a DEXA T-score less than −2.5 at one wrist and one hip, indicative of osteoporosis, took one of the above tablets per day for 12 weeks and no other medication.

Blood samples were taken before the first day (Day0) and on the last day (Day84). The blood samples taken were analysed for the well known and accepted bone resorption marker assay based on specific antibodies raised against isolated collagen peptides containing cross-links, in this case the beta-isomer of C-telopeptide of type 1 collagen known as CTX Crosslaps.

At Day84 all ten women showed a reduction in the rate of bone resorption of between 10% and 43% compared with Day0.

The blood samples were also analysed for total procollagen type 1 N-terminal propeptice (P1NP) the marker for the rate of bone formation.

At Day84 all ten women showed a reduction in P1NP of between 8% and 23% compared with Day0.

The reduction in bone resorption marker and in bone formation marker provided by the present invention shows a significant improvement in bone health as indicated by the two markers.

None of the ten participants reported any side effects at as a result of taking the tablets as described. Side effects are most unlikely since the contents of the tablets are unchanged by being used and no substance escapes into the body.

The blood markers CTX Crosslaps and P1NP are the two markers recommended for use in clinical trials by the International Osteoporosis Foundation (DOI 10. 1515/CCLM.2011.602).

1. A method for treating osteoporosis comprising administering to a subject in need thereof at least one substance selected from one or more of the group consisting of calcium phosphate, sodium chloride, potassium chloride, magnesium chloride, calcium carbonate, and sodium bicarbonate, the substance being coated with an agent selected from the group consisting of a ceramic, a polymer, a natural wax, and beeswax hardened with cornstarch and talc and a combination thereof, the agent being characterized in that it forms a liquid impermeable but gas permeable layer surrounding said substance preventing the passage of said substance through said coating wherein, in-use said substance is not released into solution in the body and is released from the body intact without entering into circulation.

2. The method of claim 1, further characterized in that the coated substance is formulated as a medicament is in a form selected from the group consisting of: a pill, a tablet, a lozenge, a bolus, a capsule, a caplet, a granule and a microparticle.

3. The method according to claim 3, wherein the medicament is in granular or microparticle form, selected from the group consisting of: a suspension, a cream, a gel and a paste.

4. The method according to claim 2, whereby the medicament is prepared for use with a patch for holding said coated substance near to or against the skin of a patient.

5. The method according to claim 1 wherein said substance is calcium phosphate.
